(19) United States
(12) Patent Application Publication
(10) Pub. No.: US 2024/0350546 A1
(43) Pub. Date: Oct. 24, 2024

(54) CHIMERIC ANTIGEN RECEPTOR (CAR) T CELL THERAPY PLATFORM

(71) Applicants: The Texas A&M University System, College Station, TX (US); The Research Institute at Nationwide Children's Hospital, Columbus, OH (US)

(72) Inventors: Xiaotong Song, Pearland, TX (US); Ruoning Wang, Columbus, OH (US); Abhijit Sarkar, Houston, TX (US); Yue Hu, Beijing (CN)

(73) Assignees: The Texas A&M University System, College Station, TX (US); The Research Institute at Nationwide Children's Hospital, Columbus, OH (US)

(21) Appl. No.: 18/686,868

(22) PCT Filed: Aug. 31, 2022
(86) PCT No.: PCT/US2022/042193

§ 371 Date: Feb. 26, 2024

Related U.S. Application Data
(60) Provisional application No. 63/238,927, filed on Aug. 31, 2021.
(60) Provisional application No. 63/355,396, filed on Jun. 24, 2022.

(51) Int. Cl. A61K 35/17; A61K 39/00; A61P 35/00; C07K 14/55; C07K 14/705; C12N 5/0783; C12N 9/78

(52) U.S. Cl. CPC A61K 35/17; A61K 39/4611; A61K 39/4631; A61K 39/4644; A61P 35/00; C07K 14/55; C07K 14/70596; C12N 5/0636; C12N 9/78; C07K 2317/622; C07K 2319/02; C07K 2319/30; C12Y 305/04004

(57) ABSTRACT

The present application provides methods and compositions for treating cancers using a CAR T cell therapy platform. Also provided are methods and use of the CAR T cells for treating diseases and conditions, such as cancer, and in particular any disease or condition associated with elevated adenosine or other associate marker.

### CROSS-REFERENCE TO RELATED APPLICATIONS

This application claims priority from, and incorporates by reference the entire disclosures of, U.S. Provisional Patent Application No. 63/238,927 filed on Aug. 31, 2021 and U.S. Provisional Patent Application No. 63/355,396 filed on Jun. 24, 2022.

### TECHNICAL FIELD

CAR T cell therapy has represented an exciting breakthrough in the treatment of patients with hematologic malignancies leading to the FDA approval of CAR T cell therapies for Acute Lymphoblastic Leukemia (ALL), Chronic Lymphocytic Leukemia (CLL), lymphoma, and Multiple Myeloma (MM). However, the antitumor activity of CAR T cell therapy in solid tumors has been modest so far in clinical studies. The suboptimal efficacy of current CAR-T cell therapies for solid tumors is most likely due to i) immunosuppressive solid tumor microenvironment, ii) suboptimal T cell persistence in vivo post cell injection, and iii) suboptimal CAR-T cell trafficking in vivo. Thus, it is desirable to develop novel CAR-T cell therapy technology platforms that display enhanced cytotoxicity against tumors, specifically solid tumors.

### BACKGROUND

A key but largely overlooked problem of CAR T cell therapy for tumors, specifically solid tumors, is nutrient competition between tumor cells and T cells in the nutrient-poor tumor microenvironment (TME). The TME represents a dramatic example of metabolic stress, wherein the high metabolic demands of cancer cells can restrict the function of CAR T cells through competition for nutrients (i.e., glucose) and by producing immunosuppressive metabolites (e.g., adenosine). Therefore, in order for CAR T cell therapy to be effective, a reliable source of nutrition is needed.

Inosine is a common component of food, and studies demonstrate that inosine has neuroprotective, cardioprotective and immunomodulatory effects. Although inosine has a weak binding affinity with adenosine receptor, earlier studies have shown that inosine produces anti-inflammatory effects related to the activation of adenosine receptors, mainly the A2a and A3 receptor whose activation can contribute to the reduction of pro-inflammatory cytokines, and tissue protective effects from endotoxin-induced and 2,4,6-trinitrobenzene sulfonic acid (TNBS)-induced inflammation. However, a small body of literature has showed opposing findings that inosine analogs can be proinflammatory and that A2AR signaling can sustain Th1 and anti-tumor immunity in mice. A recent study has showed that inosine is a key bacterial-derived metabolite acting through T cell-specific A2AR signaling to promote Th1 cell activation in a context-dependent manner. Specifically, in the presence of IFN-gamma, inosine strongly boosted Th1 differentiation of native T cells, whereas in the absence of IFN-gamma, inosine inhibited Th1 differentiation. Since adoptive T cell transfer and immune checkpoint inhibitors have shown the capability of converting a suppressive TME to a supportive TME, it is reasonable that inosine can amplify the antitumor efficacy of T-cell therapy or immune checkpoint inhibitors. In addition, an independent study has shown that inosine can strongly sensitize tumor cells to the cytolytic effect of immune cells by enhancing tumor immunogenicity.

Adenosine deaminase (ADA) is known to have anti-tumor activity. Several groups have developed ADA as cancer therapy. For example, a PEG-ADA2 (Adenosine deaminase 2) has been developed and has shown antitumor activities in mouse models. ADA's half-life is only several hours long, and PEG conjugation does not significantly increase the protein's half-life. Therefore, in order for ADA to serve as an effective cancer therapy, the half-life must be improved.

ADA decreases the level of adenosine, an immunosuppressive metabolite, by irreversibly converting it to inosine. ADA is a key enzyme of purine metabolism required for the breakdown of adenosine from food and for the turnover of nucleic acids in tissues. ADA1 and ADA2 are two ADA isoenzymes in humans. ADA1 is primarily involved in intracellular activity in most body cells, particularly lymphocytes. ADA1 deficiency in humans causes severe combined immune deficiency (SCID). ADA2 is the predominant form present in human blood plasma. ADA1 and ADA2 might play different roles in regulating immune responses independent of their ADA activity. Studies have shown that ADA1 has co-stimulatory effects on T cell-mediated immunity by binding to Teffs and CTLs that express CD26. In contrast, ADA2 binds to neutrophils, CD16+ monocytes, NK cells, B cells and CD39+ Treg that do not express CD26. Recent studies have shown that in mice, PEGylated ADA2 inhibited tumor growth by targeting adenosine in an enzyme activity dependent manner and modulating immune responses. Additionally, ADA1 has a 100-fold higher affinity for adenosine than ADA2.

Adenosine signaling has emerged as a key immuno-metabolic checkpoint in tumors. ATP or NAD+ accumulate within the TME and can generate adenosine by ecto-nucelotidases including CD39, CD73, CD38, CD203a, ALP and PAP53-61. Studies have shown that adenosine enables tumor cells to escape immune-surveillance by suppressing the function of multiple potentially protective immune cells including T cells, DCs, NK cells, macrophages and neutrophils, while enhancing the activity of immunosuppressive cells such as MDSCs and Tregs. In addition, adenosine activates cancer associated fibroblasts and induces the formation of new blood vessels.

Several agents such as small molecules or mAbs targeting CD73 and CD38 to limit its production or targeting A2AR and A2BR to limit its binding to immune cells have been developed, and pre-clinical studies have demonstrated their anti-tumor activity, alone and in combination with other immunotherapies including immune checkpoint inhibitors and adoptive cell transfer. However, early phase clinical studies show that their antitumor effects are suboptimal. This can be explained by the fact that multiple ectonucleotidases contribute to extracellular adenosine production and adenosine binds to multiple receptors such as A2AR, A2BR and A3R to suppress antitumor immunity.

Although cure rates for several malignancies have significantly improved, the outcome for patients with advanced solid tumors remains grimly unchanged over the last decades, underscoring the need for new therapies. Oncolytic vaccinia viruses are an appealing addition to the current treatment options of solid tumors because of their safety and their potential to infect, replicate in, and lyse tumor cells. Although clinical studies have shown that intratumoral or intravenous injection of oncolytic vaccinia viruses was safe and can induce tumor lysis, the antitumor efficacy of the oncolytic vaccinia viruses is suboptimal and most tumors occurred. This is most likely due to the limited activation of antitumor T-cell responses within the tumor of oncolytic vaccinia virus and the immunosuppressive tumor environment. Therefore, an oncolytic vaccinia virus that 1) reverses immunosuppressive tumor microenvironment, and 2) effectively supports T-cell function, will overcome the current limitations of oncolytic vaccinia viruses.

There is increasing evidence that T cells are able to control tumor growth and survival in cancer patients, both in early and late stages of the disease. For example, adoptive transfer of T cells has been demonstrated to effectively treat disseminated tumors including Hodgkin's Lymphoma, nasopharyngeal carcinoma, neuroblastoma and melanoma. However, tumor-specific T-cell responses are difficult to mount and sustain in cancer patients and are limited by numerous immune escape mechanisms of tumor cells selected during immunoediting. Therefore, it would be desirable to develop an alternative strategy to utilize T cells for cancer therapy with the ability of overcoming the immune escape mechanism of tumors.

The present application addresses the disadvantages that exist in current cancer therapies. Methods and compositions for treating cancers using an engineered immune cells (e.g., CAR-T cells, CAR-NK cells, CAR-NKT cells, macrophage) or a recombinant oncolytic virus encoding a adenosine deaminase are provided. The engineered immune cells or recombinant oncolytic virus encodes one or more other heterologous proteins. The present application also provides methods and uses of the engineered immune cells or oncolytic virus for treating diseases and conditions, such as cancer, and in particular any disease or condition associated with elevated adenosine or other associate marker.

### SUMMARY OF THE INVENTION

An embodiment of the claimed invention is directed to an engineered cell comprising a nucleotide sequence encoding an Adenosine deaminase (ADA). In certain embodiments, the cell is T cell, natural killer cell, natural killer T cell, dendritic cell, macrophage, mesenchymal stem cell, and derivatives thereof.

An embodiment of the claimed invention is directed to a pharmaceutical composition comprising an engineered cell.

A further embodiment of the claimed invention is directed to a method of administering a pharmaceutical composition comprising an engineered cell to a patient in an amount effective for treatment of a cancer.

Another embodiment of the invention is directed to a recombinant oncolytic virus comprising a nucleotide sequence encoding an ADA, wherein the nucleotide sequence encoding the ADA is operably linked to a promoter.

An embodiment of the invention is directed to a recombinant protein comprising an amino acid sequence encoding an ADA.

### BRIEF DESCRIPTION OF THE DRAWINGS

FIG. 1 shows the designs of conjugated ADA used in embodiments of the invention;

FIG. 2 shows a rationale for an CD26/ADA-CD3-scFv expressed CAR T cell therapy. CAR T cells are engineered to express a tumor-specific CAR, a membrane CD26, and a secreted bispecific ADA-CD3-scFv. These CAR T cells 1) target and kill specific tumor cells; 2) stimulate CAR T cells by ADA-CD3-scFv (CD3-scFv anchors ADA to T cells since the binding affinity of CD3 and CD3-scFv is higher than that of ADA-CD26, while CD3-scFv itself can activate T cells), inducing CAR T cell activation and migration, 3) release CAR T cells from adenosine-mediated immune suppression; and 4) provide inosine as alternative carbon source for CAR T cells, and enhance the anti-tumor activity of CAR T cell therapy;

FIG. 3 shows the construction of an HER2-targeted CD26/ADA-CD3-scFv expressed CAR retroviral vector and an CD26/ADA-CD3-scFv expressed retroviral vector;

FIG. 4 shows that Rv-CD26 transduced CAR T cells can resist the suppression of TGF-beta on CD26 expression, while CD26 expression of non-Rv-CD26-transduced CAR T cells were suppressed by TGF-beta;

FIGS. 5A and 5B show that Rv-CD26 transduced CAR T cells displayed enhanced migration capacity in a fluorescent migration assay (FIG. 5A) and a transwell migration assay (FIG. 5B);

FIG. 6 shows the mechanism of action of the ADA-CD3-scFv on the metabolic reprogrammed CAR-T cells: (A) CD3-scFv anchors ADA to T cells since CD3 and CD3-scFv has higher binding affinity than that of CD26-ADA, while CD3-scFv can directly activate T cells through CD3; (B) ADA engages CD26, co-stimulating CAR T cells; and (C) ADA converts ADO to INO, overcoming ADO-mediated immunosuppression on CAR T cells while providing INO as alternative energy resources for CAR T cell survival and proliferation;

FIGS. 7A and 7B show the expression of ADA-CD3 (ADA-CD3-scFv) by the retroviral vector transduced HEK 293 T or Jurkat T cells: (FIG. 7A) western blotting of ADA-CD3-scFv using monoclonal Ab against ADA in HEK 293 T cells that were transduced with retroviral vector expressing ADA-CD3-scFv with or without a human IL2 signal peptide compared to non-transduced HEK 293 T cells, and (FIG. 7B) western blotting of ADA-CD3-scFv using monoclonal Ab against ADA in Jurkat T cells that were transduced with retroviral vector expressing ADA-CD3-scFv with a human IL2 signal peptide or a lentiviral vector expressing CRISPR ADA activation elements, compared to non-transduced Jurkat T cells;

FIGS. 8A, 8B and 8C show the expression of ADA-CD3-scFv (ADA-CD3): (FIG. 8A) ELISA measurement of ADA in the supernatant or cell lysate of Rv-ADA-CD3-IL2sp (signal peptide)-transduced, Rv-ADA-CD3 (no IL2 sp)-transduced or non-transduced HEK 293 T cells; (FIG. 8B) ELISA measurement of ADA in the supernatant of co-culture of Jurkat T cells with Rv-ADA-CD3-IL2sp (signal peptide)-transduced, Rv-ADA-CD3 (no IL2 sp)-transduced or non-transduced HEK 293 T cells at day 1 and day 4; and (FIG. 8C) flow analysis of ADA-CD3 on Jurkat T cells or Rv-CD26-transduced Jurkat T cells post co-culture with Rv-ADA-CD3-IL2sp (signal peptide)-transduced, Rv-ADA-CD3 (no IL2 sp)-transduced or non-transduced HEK 293 T cells;

FIGS. 9A, 9B and 9C show the enzyme activity of ADA (ADA1) by ADA enzyme activity assay: (FIG. 9A) ADA enzyme activity assay of co-culture of Jurkat T cells and Rv-ADA-CD3 (with IL2sp)-transduced or non-transduced HEK 293 T cells (Top: original reads; Bottom: non-transduced HEK 293 T cell background reads were subtracted); (FIG. 9B) ADA binding assay of Jurkat-Dual or Jurkat-Dual-CD26 T cell lines; and (FIG. 9C) ADA binding assay of Jurkat-NEFA or Jurkat-NFAT-CD26 T cells lines;

FIGS. 10A, 10B, 10C and 10D show T-cell activation using Jurkat NFAT reporter T cell line: (FIG. 10A) Jurkat NFAT reporter T cells were added with the supernatant of or the Rv-ADA or Rv-ADA-CD3 transduced HEK 293 T cell for 6 hours and subjected to luciferase assay; (FIG. 10B) Jurkat NFAT reporter T cells were added with the supernatant of or the Rv-ADA or Rv-ADA-CD3 transduced HEK 293 T cell for 24 hours and subjected to luciferase assay; (FIG. 10C) Jurkat NFAT reporter T cells were added with the supernatant of or the Rv-ADA-CD3 or Rv-ADA-CD3 (IL2sp) transduced HEK 293 T cell for 6 hours or 24 hours and subjected to luciferase assay; and (FIG. 10D) ADA-CD3 (IL2sp) expression level were calculated;

FIGS. 11A, 11B, 11C, 11D and 11E show the antitumor activity of ADA-CAR-T cells using in vitro LDH assay: (FIG. 11A) GPC3 CAR expression level of different groups of cells by flow analysis; (FIG. 11B, FIG. 11C, FIG. 11D and FIG. 11E) LDH assay of groups of CAR T cells against GPC-positive HepG2 or Huh7 liver tumor cell lines; and

FIGS. 12A, 12B, 12C and 12D show the results of an antitumor efficacy and toxicity study carried out in mice.

### DETAILED DESCRIPTION

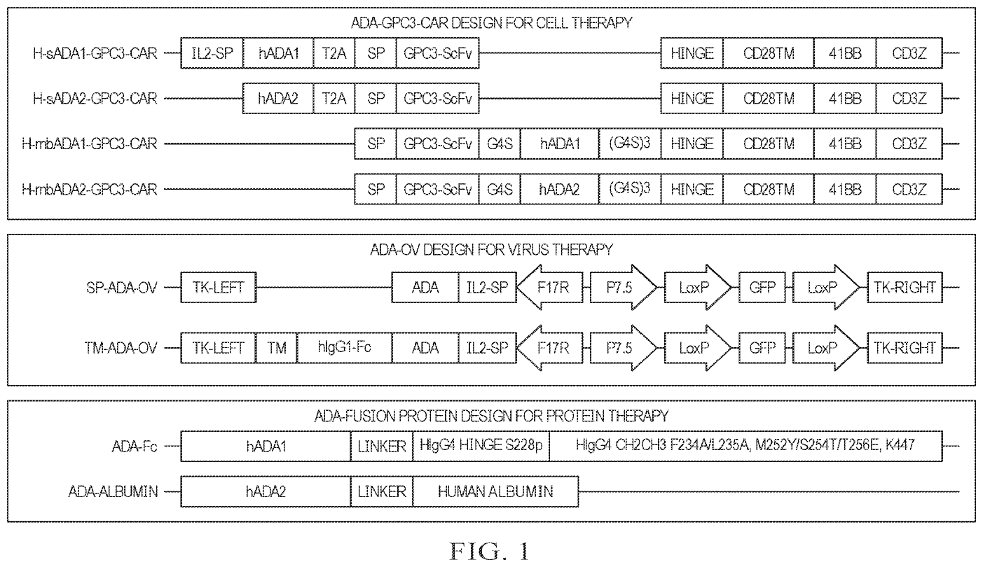

An embodiment of the invention is directed to an engineered CAR T cell that expresses a tumor-specific CAR and expresses ADA (FIGS. 1 & 3). The engineered CAR T cell 1) targets and kills tumor-antigen specific tumor cells; 2) CAR T cells are co-stimulated by ADA through CD26 signaling, 3) CAR T cells are released from adenosine-mediated immune suppression; and 4) inosine is provided as an alternative carbon source for CAR T cells, enhancing the anti-tumor activity of CAR T cell therapy.

Studies have shown that extracellular adenosine in the TME has a marked suppressive effect on immune responses, suppressing T cell function and stabilizing immunosuppressive regulatory cells. Thus, ADA that irreversibly converts adenosine to inosine release CAR T cells from adenosine-driven immunosuppression, while providing an energy source for CAR T cells. In addition, ADA mediates effective co-stimulatory signals and promote T-cell proliferation independent of its enzyme activity. FIG. 1 sets forth examples of ADA-CAR T cell conjugates.

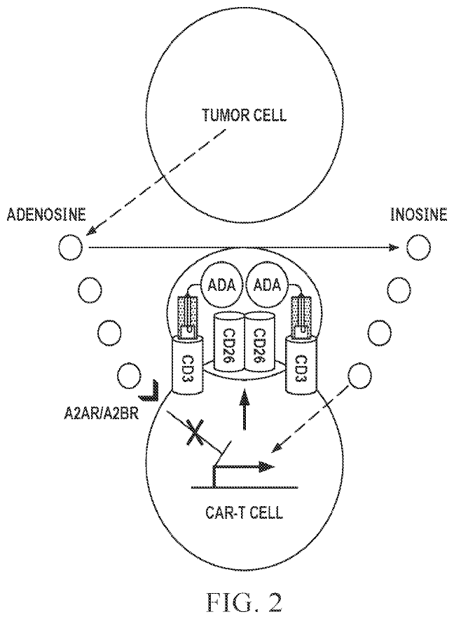

A schematic of an ADA-CD3-scFv/CD26 overexpressed metabolic reprogrammed (MR) CAR-T cell therapy platform is shown in FIG. 2. ADA-CD3-scFv/CD26-MRCAR T cells that express a tumor Ag-specific CAR and ADA-CD3-scFv/CD26 complex enhance the anti-tumor activity of CAR T cell therapy through a series of mechanisms: i) CAR molecule targets and direct T cells to kill tumor Ag-positive tumor cells; ii) ADA/CD26 complex releases the adenosine-mediated immune suppression by providing inosine as carbon source for CAR T cells by converting adenosine to inosine in the TME; and iii) CD26 mediates CAR T cell trafficking and remain bound to T cell surface via ADA. Such an ADA-CD3-scFv/CD26-MRCAR T cell has the potential to overcome current limitations on CAR-T cell therapy against solid tumors.

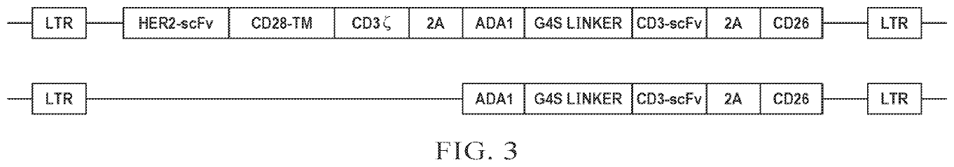

The ADA-CD3-scFv/CD26-MRCAR in FIG. 3 contains the following components: a chimeric antigen receptor containing a tumor antigen (e.g., HER2, GPC3) specific scFv (small chain variable fragment), a spacer, hinge, transmembrane domain (e.g., CD28 and/or 4-1BBL), CD3 zeta domain; ADA-CD3-scFv bispecific fusion protein, and CD26. These components were linked with the 2A sequence, or expressed in separate viruses (e.g., retroviral vector, lentiviral vector). Ecto-ADA is critical for protecting T cells from adenosine-mediated immune suppression, while cell free ADA is not. Thus, it is desirable to develop an effective strategy to anchor ADA on the surface of T cells. Rv-ADA T cells can express ˜ng ADA/ml per 24 hours and the ADA expression by Rv-ADA T cells only increases by 20% compared to that of non-modified T cells. However, ADA concentration in human breast tumor tissue, normal tissue, or serum is 16.4-47.7 ug/g, 11.5 ug/g, or 160-242 ng/ml respectively. Thus, ADA-armed T cell therapy is significantly limited by the ADA expression level of T cells. Anchoring ADA to the cell surface overcomes this limitation by engagement of CD26 on the surface of T cells.

In certain embodiments, the CAR comprises an amino acid sequence having at least 70%, or at least 75%, or at least 80%, or at least 85%, or at least 90%, or at least 95%, or 100% sequence identity to an amino acid sequence selected from the group consisting of SEQ ID Nos: 1, 3, 5, 7, 9, 18, 20, 22, and 24. 10, 12, 14, and 16 2, 4, 6, 8, 9, 19, 21, 23, and 25 11, 13, 15, and 17

As shown in FIG. 2, CD3-scFv engages with CD3 on the infused CAR-T cells or endogenous T cells by anchoring ADA on T cell surface and promoting ADA-CD26 engagement and activating T cells through CD3 signaling. When ADA engages with CD26, it activates the infused CAR-T cells or endogenous T cells. ADA converts adenosine to inosine, overcoming adenosine-mediated immune suppression on CAR-T cells and providing inosine as alternative energy resources for CAR-T cells.

FIG. 3 also shows a vector that contains ADA-CD3-scFv bispecific fusion protein and CD26 only. This vector can be used together with a vector that expresses a CAR against any type of tumor antigens to transduce human T cells to produce MRCAR T cells.

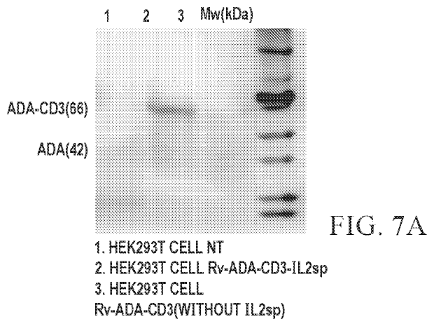

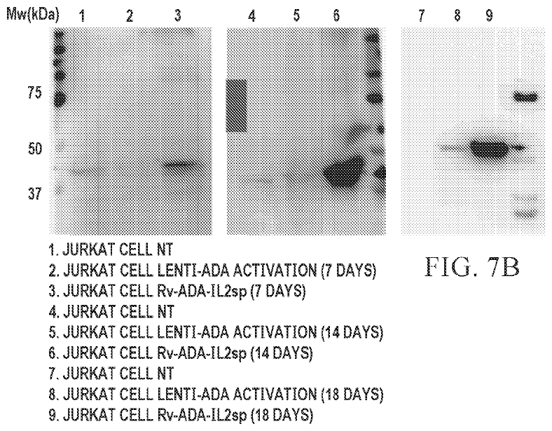

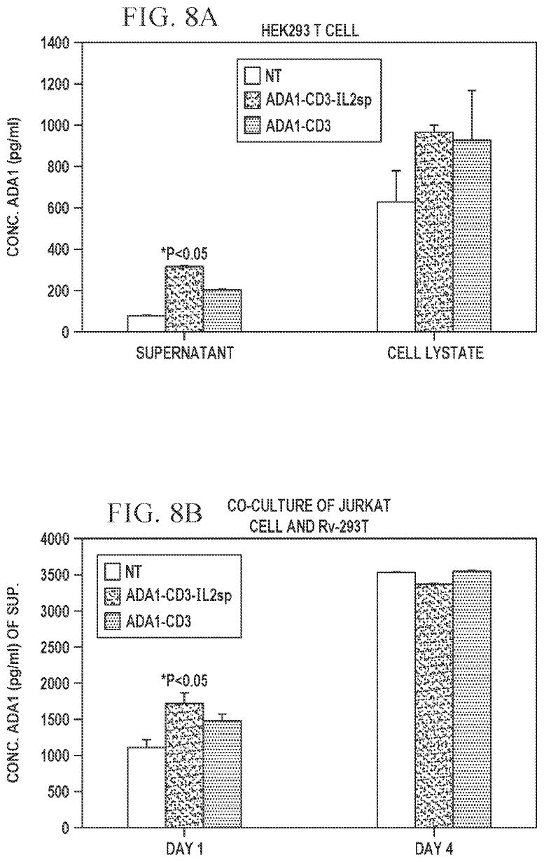

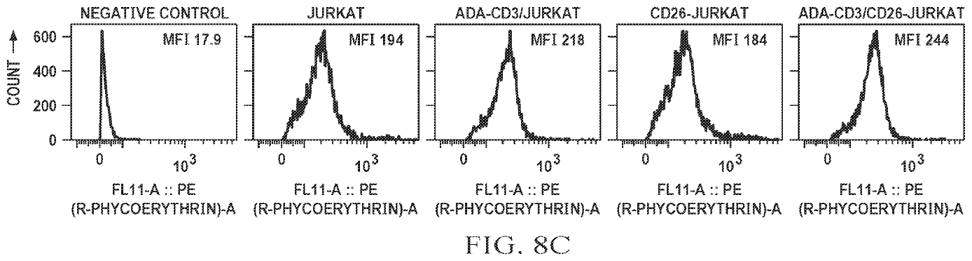

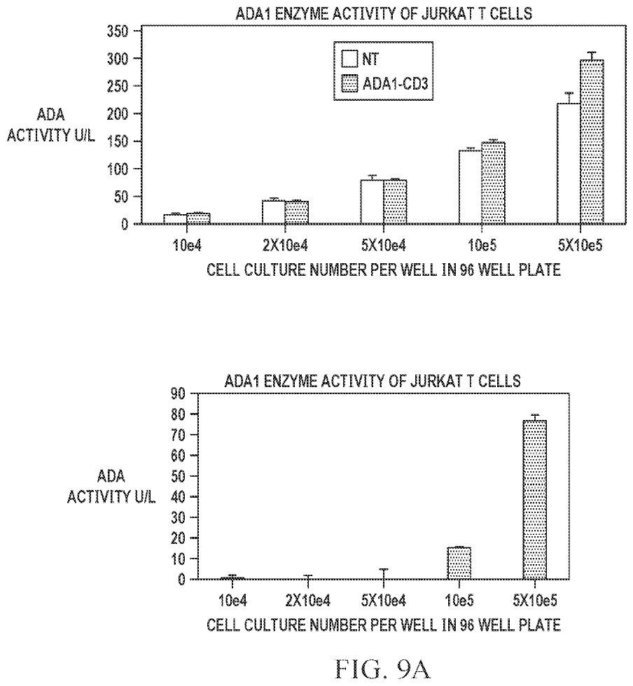

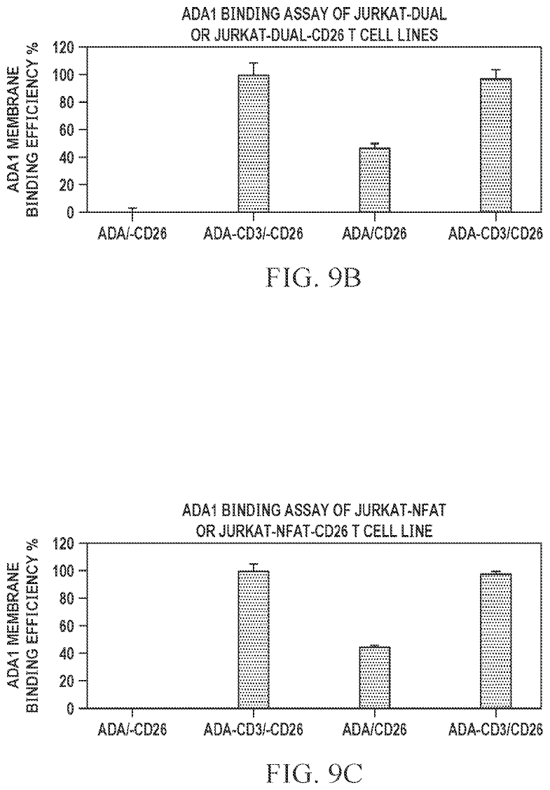

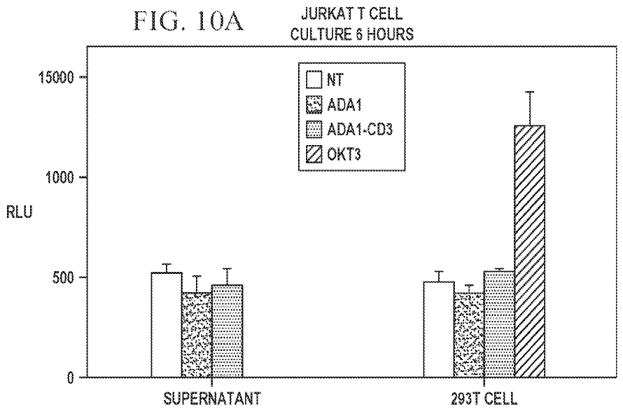

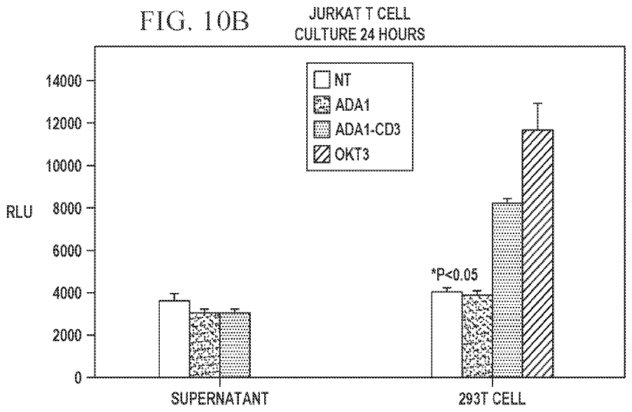

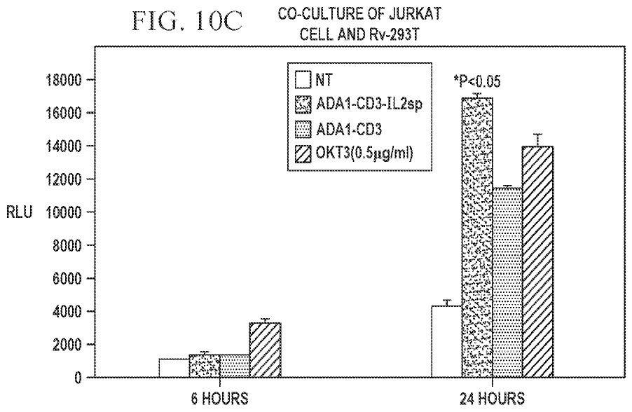

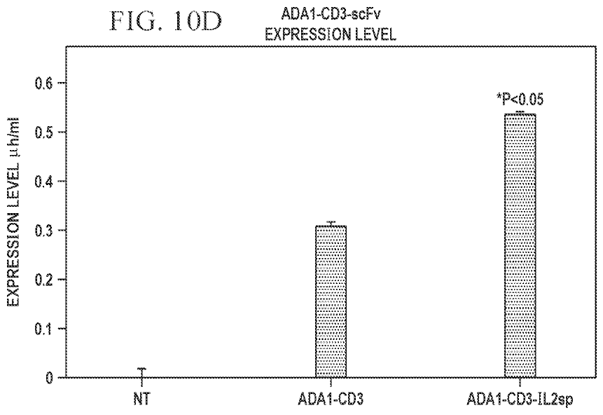

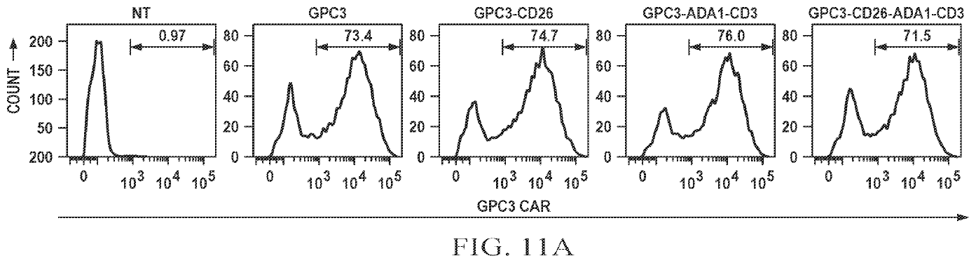

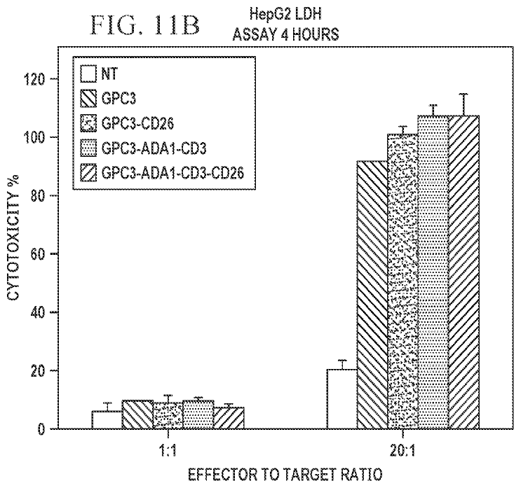

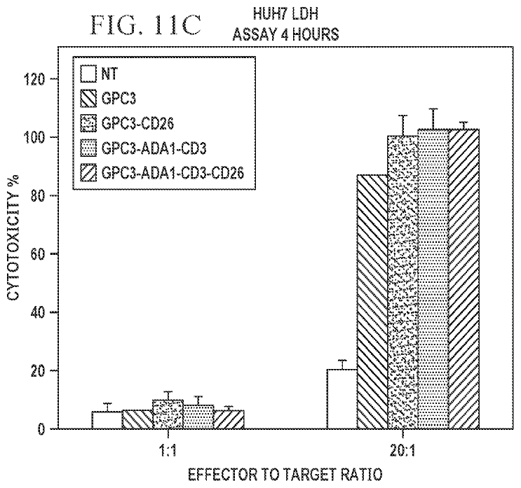

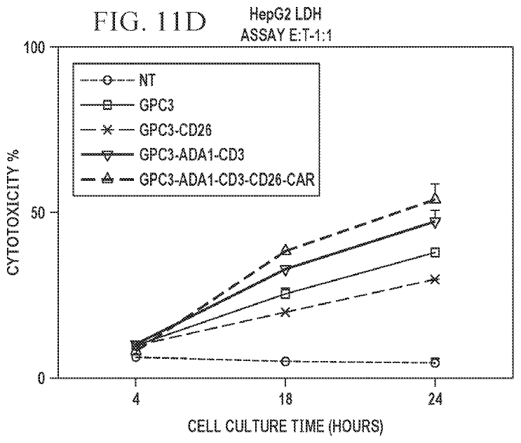

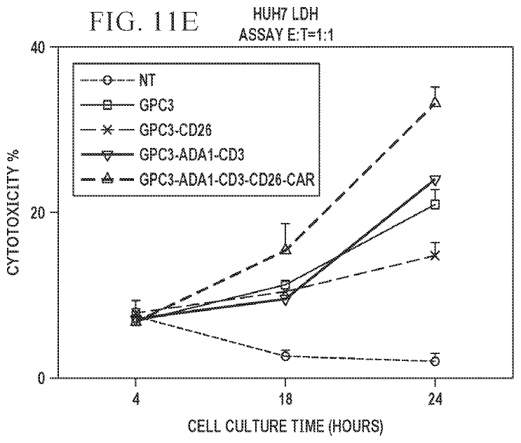

A retroviral vector system is used to express a HER2-specific CAR, a membrane CD26 and a secreted ADA-CD3-scFv (FIG. 3). A retroviral vector system was also used to express a membrane CD26 and a secreted ADA-CD3-scFv only (FIG. 3). T cell codon optimization was used to enhance transgene expression of ADA in human T cells. An optimized human IL2 signal peptide was added to express ADA by human T cells. Human peripheral monocytes were activated by plate-bound CD3 and CD28 antibodies for 2 days followed by virus transduction. Expression and secretion of ADA1 by T cells was confirmed by western blotting (FIG. 7) and ELISA (FIG. 8). ADA expression effectively converted adenosine to inosine (FIG. 9). As shown in FIG. 10, ADA-CD3 expression effectively activated human T cells (FIG. 10). The in vitro LDH cytotoxicity assays showed that GPC3-specific MR-CAR displayed enhanced cytotoxicity against GPC3-positive liver cancer cell lines such as HepG2 and Huh7 (FIG. 11).

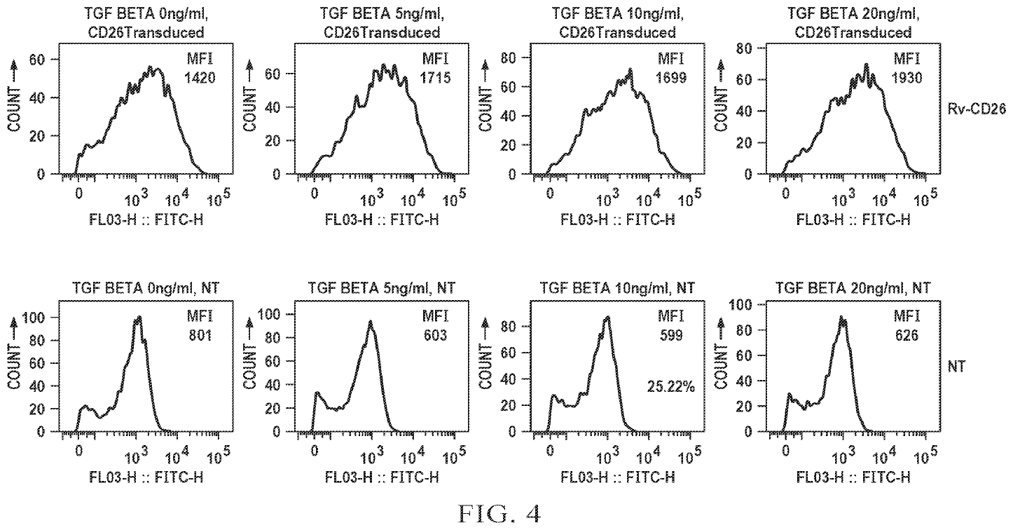

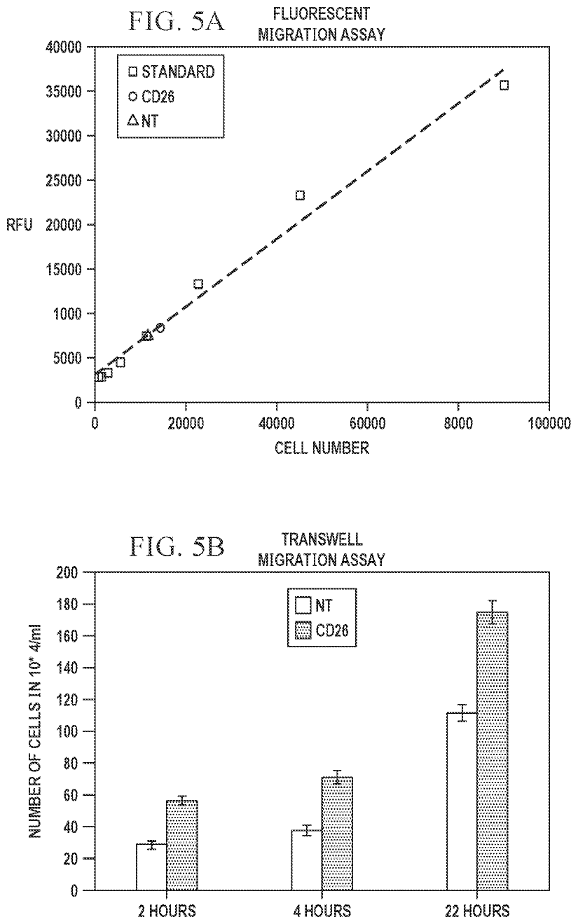

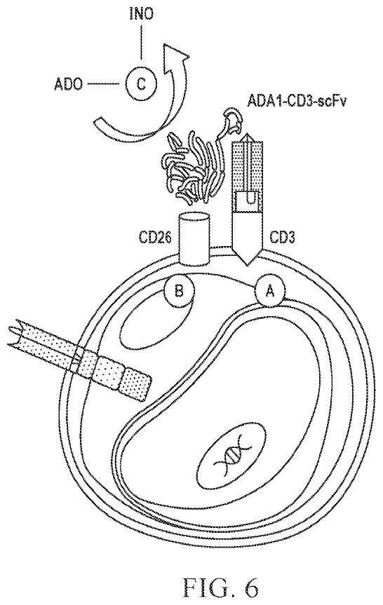

Overexpression of CD26 in CAR T cells is critical for CAR T cell therapy. As shown in FIG. 4, in the presence of TGF-beta, the expression of CD26 on CAR T cells are reduced in 48 hours, suggesting the CD26 expression can be suppressed by tumor microenvironment. Rv-CD26 can restore the CD26 expression in CAR T cells. As shown in FIG. 4, Rv-CD26 transduced T cells expressed CD26 at a higher level compared to non-modified T cells. In addition, Rv-CD26 transduced T cells resisted TGF-beta-mediated immunosuppression and retained the CD26 expression at high level. In addition, Rv-CD26 transduced T cells had enhanced migration capacity, as evidenced by in vitro florescent migration assay and transwell migration assay (FIG. 5).

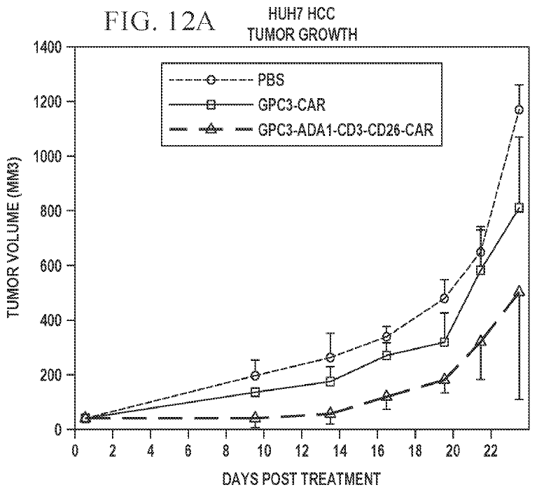

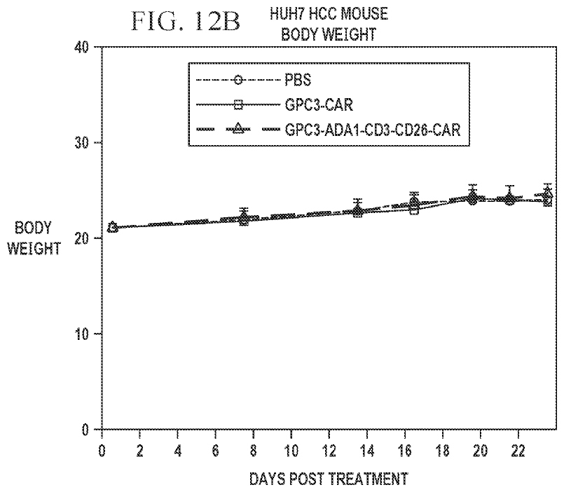

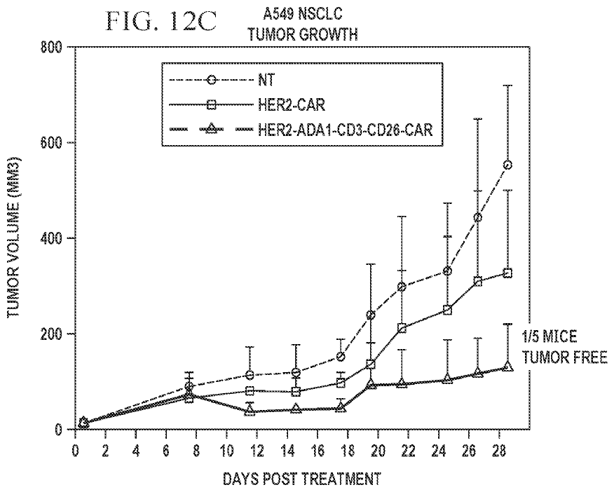

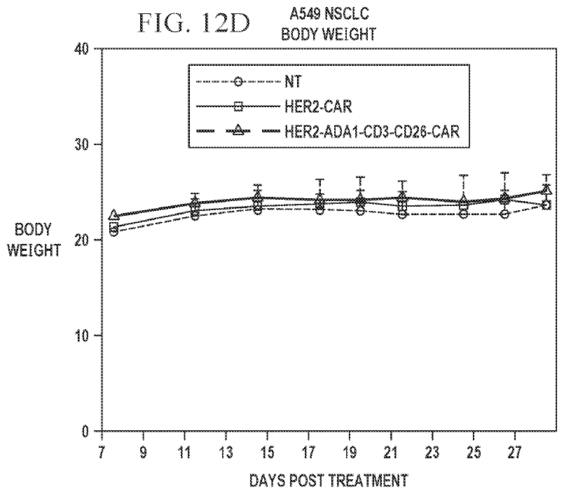

A further embodiment of the invention is directed to an ADA-CAR T cell therapy in transplantable mouse tumor models. The results show the efficacy and antitumor properties of a CAR T cell therapy model. MR-CAR T cells displayed enhanced anti-tumor effects in two mouse models. In the first model, NSG mice were inoculated with 2×10^{e6} Huh7 HCC tumor cells on the right flank at day 0. At day 7, the Huh7 tumor bearing mice were treated through tail vein with PBS, 2×10^{e6} GPC3-CAR T cells, or 2×10^{e6} GPC3-ADA1-CD3-CD26-CAR (designated as GPC3-MR-CAR), followed by tumor monitoring with caliper (FIG. 12A) and body weight measurement (FIG. 12B). In the second model, NSG mice were inoculated with 2×10^{e6} A549 NSCLC tumor cells on the right flank at day 0. At day 7, the A549 tumor bearing mice were treated through tail vein with PBS, 2×10^{e6} HER2-CAR T cells, or 2×10^{e6} HER2-ADA1-CD3-CD26-CAR (designated as HER2-MR-CAR), followed by tumor monitoring with caliper (FIG. 12C) and body weight measurement (FIG. 12D). In both models, MR-CAR T cells showed enhanced anti-tumor activity. MR-CAR T cells significantly inhibit tumor growth in both Huh7 HCC and A549 NSCLC mouse models while native CAR T cells only moderately inhibit Huh7 or A549 tumor growth. In addition, no significant difference in body weight was observed in either Huh7 HCC or A549 NSCLC mouse model, indicating MR-CAR T cells did not induce toxicity in the mouse models. The promising results in the mouse models provide a good predictor of efficacy of the CAR T cell platform in human subjects.

Another embodiment of the invention is directed to an optimized ADA that displays enhanced ADA activity compared to native ADA. In certain embodiments, the optimized ADA is an ADA-Fc fusion protein that significantly prolongs ADA's half-life, and improves its antitumor activity (FIG. 1). In other embodiments, the optimized ADA is an antibody conjugated ADA (FIG. 1).

Another embodiment of the invention is directed to an engineered oncolytic virus that expresses ADA or ADA derivatives such as ADA-Fc (FIG. 1). The engineered oncolytic virus that expresses ADA will infect and replicate in the tumor cells, and expresses ADA or ADA-Fc that can convert ADO to INO within tumor tissues, enhancing the antitumor efficacy of oncolytic virus.

In certain embodiments, the engineered oncolytic virus comprises an amino acid sequence having at least 70%, or at least 75%, or at least 80%, or at least 85%, or at least 90%, or at least 95%, or 100% sequence identity to an amino acid sequence selected from the group consisting of SEQ ID NOS: 5, 7, 9, 22, and 24.

### WORKING EXAMPLES

### CAR T Cell Generation

### Material:

OKT3 (ortho Biotech)—1 mg/ml
CD28 (no azide (NA)/low endotoxin (NE) (Pharmingen #33740D)—1 mg/ml
Plate: Non-tissue culture treated 24-well plate (Falcon #1147)

### Protocol:

### Day 0:

1. Dilute antibodies to a final concentration of 1 ug/ml in sterile water (for example to coat 6 wells, add 3 μL of OKT3 and 3 μL of CD28 to 3 ml of sterile water)
2. Add 0.5 ml (=0.5 ug of antibody) of the antibody solution/well in the 24 well plate; seal it with parafilm
3. Incubate at +4° C. overnight

### Day 1:

1. Resuspend 5×10⁶ PBMC in 10 ml of complete RPMI (10% FCS)
2. Aspirate antibody solution and wash wells once with RPMI or DMEM complete
3. Aspirate media and plate 1×10⁶ PBMC/well in a final volume of 2 ml of complete media

### Day 2:

1. Aspirate and discard about 1 ml from each well and add back 1 ml of complete RPMI medium containing 200 U/ml of IL-2
2. Coat a non-tissue culture treated 24-well plate with retronectin; seal it with parafilm
3. Place the retronectin coated plate at +4° C. overnight.

### Day 3:

1. Remove retronectin solution and wash wells once with RPMI or DMEM complete (please store retronectin at +4 C in 15 cc tube; can be reused once within one month)
2. Add 500-700 ul of viral supernatant to well. Place plate into incubator for 30 min
3. Remove viral supernatant; repeat step 2
4. During incubation time prepare blasts at 5×10⁵ cells/cc with 400 U/cc IL-2
5. Remove viral supernatant. Add 1.5 cc of viral supernatant.
6. Add 500 ul of blasts (2.5×10⁵ cells)
7. spin the plate at 1000 g for 30 minutes then put the plate back in the incubator (optional)

### Day 5/6:

1. Remove 1 cc of media from wells; transfer cells of retronectin-coated plate into new well (by scraping); add 1 cc of complete media containing 100 U/ml of IL-2
2. Feed with IL-2 (100 U/ml) every 2-3 days. Freeze back or use in experiments with in 7 to 21 days.
   MR-CAR Resisted TGF-Beta Mediated Suppression on CD26 Expression on CAR T Cells and Displayed greater migration capacity.

Human PBMC was activated by CD3/CD28 antibodies followed by transduced with Rv-CD26 as described as above. The Rv-CD26 transduced T cells (Rv-CD26) or non-transduced T cells (NT) were cultured in the absence or presence of 5 ng/ml, 10 ng/ml or 20 ng/ml of TGF-beta for 48 hours. The T cells were collected and stained with FITC-conjugated human CD26 specific antibody. The CD26 expression on T cells was then detected by flow analysis and showed as MFI (median fluorescence intensity). Rv-CD26 transduced human T cells expressed CD26 at a higher level compared to non-transduced T cells (FIG. 4, Rv-CD26 vs NT=MFI 1420 vs MFI 801) in the absence of TGF-beta. When NT T cells were cultured in the presence of TGF-beta (5 ng/ml, 10 ng/ml, or 20 ng/ml) for 48 hours, CD26 expressions were decreased from MFI 801 to MFI 603, MFI 599, or MFI 626 individually, while CD26 expression on Rv-CD26-transduced T cells remain unchanged after 48 hours culture in the presence of TGF-beta (5 ng/ml, 10 ng/ml, or 20 ng/ml). These results suggested that Rv-CD26 transduced CAR T cells can resist the suppression of TGF-beta on CD26 expression, while CD26 expression of non-Rv-CD26-transduced CAR T cells were suppressed by TGF-beta.

### To Investigate the Migration Capacity of the Human T Cells, In Vitro Transwell Migration Assay and Fluorescent Migration Assay Were Conducted.

### A. Transwell Cell Migration Assay Protocol (By Counting the Cell):

1. Grow enough cells (Rv-CD26-transduced HER2 CAR T cells or control HER2 CAR T cells) to perform a Cell Migration Assay in desired media and culture conditions.
2. Collect the cells and resuspend the cells in serum-free media and count the number of cells using a hemocytometer. Resuspend cells at 1.5×10⁶ cells/mL in a serum-free media or make the cell as per your desired concentration.
3. Bottom Chamber: Add 235 μL of HER2-positive A549 cell supernatant (Chemoattractant) and 365 μL of serum free media to make it 600 μL to the bottom chamber for both NT (Non-Transduced) and CD26 Transduced HER2-CAR T cells.
4. Place the top chamber (Transwell, 5 mm) carefully into the bottom chamber so that no air bubbles are trapped between the top and the bottom chamber.
5. Top Chamber: Add 300 μL (5,00000 cells) of cell suspension (both NT and Rv-CD26 transduced) to each well of the top chamber.
6. Carefully place the plate cover and incubate the Cell Migration Chamber at 37° C. in CO2 incubator for 2-24 hours.
7. Count the cells using a haemocytometer at different time point (2, 4, 22/24 hrs).
   B. Fluorescent Cell Migration Assay Protocol (By Fluorescent Intensity With Abcam Transwell Migration Assay Kit ab235696):

### Standard Curve:

1. Each cell type requires a separate Standard Curve. Prepare a Standard Curve by adding 50 μL cell suspension (1.8×10⁶ cells/mL, 90,000 cells or make it 50000 with 1×10⁶ cells/ml).
2. Serially dilute the cells 1:1 in Wash Buffer and generate a Standard Curve of cells (90000, 45000, 22500, 11250, 5625, 2812, 1406 and 703) in 100 μL total volume in 96 well white plate.
3. As blank, use 100 μL of Wash Buffer.
4. Add 10 μL of Cell Dye to each well.
5. Incubate at 37° C. for 1 hour.
6. Read the fluorescence at Ex/Em=530/590 nm.
7. Plot the Standard Curve of Number of Cells vs RFU obtained.
8. Fit the data points using a linear trendline.

### Count Migrated Cells:

1. Make the cell dye solution as desired depending on the number of wells. Add 100 μL Cell Dye to 1 ml Cell Dissociation Solution. Mix well.
2. Add 550 μL of the mix to each well of bottom chamber (after 22hrs, step A6). It can be after 2 Hrs or 4 Hrs too depending on experiment design.
3. Incubate at 37° C. in CO2 incubator for 60 minutes.
4. After incubation transfer 110 μL of mix from bottom chamber to the 96 well white plate. Read the plate at Ex/Em=530/590 nm. Multiply the reading by 5 to account for the 5× higher volume in each well of the 24-well plate.

### Data Analysis:

The number of cells migrated using the equation of the straight line obtained from the Standard Curve was calculated

As shown in FIG. 5, both fluorescent migration assay and transwell migration assay results showed that Rv-CD26 transduced HER2 CAR T cells displayed enhanced migration capacity compared to HER2 CAR T cells.

### ADA-CD3-scFv Expression by MR-CAR T Cells is Biologically Functional

Western blotting was used to measure the expression of ADA-CD3 (scFv) in FIG. 7. Rv-ADA-CD3 (scFv) or Rv-ADA-CD3 (scFv)-IL2sp (a human IL2 signal peptide was inserted to the 5′ terminal of ADA-CD3 (scFv) in order to increase its expression) was transduced into 293 T cells and cell lysates are subjected to western blotting using anti-ADA antibody (FIG. 7A). The results showed that Rv-ADA-CD3-IL2sp expressed ADA-CD3 at a higher level compared to Rv-ADA-CD3 (without IL2sp). Then Rv-ADA-CD3-IL2sp was compared to a lentiviral vector that expresses an ADA CRISPR activation element. Lv-ADA (CRISPR activation) virus or Rv-ADA-IL2sp were transduced into Jurkat T cells. Lv-ADA (CRPSPR activation) transduced Jurkat T cells were selected by puromycin. ADA expression was measured by western blotting using anti-ADA mAb at 7, 14, or 18 days of T cell culture. As shown in FIG. 7B, Rv-ADA1-IL2sp expressed ADA at a higher level compared to Lenti-ADA activation. These results supported that Rv-ADA-CD3-IL2sp is an optimized strategy to express ADA-CD3 (scFv) in CAR T cells.

The secretion of ADA-CD3 (scFv) by MRCAR T cells were measured by ELISA against human ADA. As shown in FIG. 8A, ADA in the supernatant or cell lysate of Rv-ADA-CD3-IL2sp (signal peptide)-transduced, Rv-ADA-CD3 (no IL2 sp)-transduced or non-transduced (NT) HEK 293 T cells were measured by ELISA. The results showed in both supernatant or cell lysate, Rv-ADA-CD3-scFv-IL2sp (ADA1-CD3-IL2sp) or Rv-ADA-CD3-scFv (no IL2sp, ADA1-CD3) expressed ADA-CD3. And Rv-ADA-CD3-IL2sp expressed a higher level of ADA-CD3 than Rv-ADA-CD3.

Then the Rv-ADA-CD3-IL2sp or Rv-ADA-CD3 transduced HEK 293 T cells were co-cultured with Jurkat T cells to mimic a stress condition for cell culture (increased cell density and cell contact) in order to confirm the ADA-CD3 expression. At day 1 or day 4 of the co-culture, the cell culture medium was collected and subjected to ELISA measurement of ADA. The results showed Rv-ADA-CD3-IL2sp induced higher ADA expression than NT or Rv-ADA-CD3. We observed that NT groups had ADA expression (FIG. 8B). This can be explained by background expression of ADA by Jurkat T or HEK 293 T cells.

Next, we investigated if the ADA-CD3 will engage CD3-expressed Jurkat T cells or Rv-CD26-transduced CD3/CD26 double positive CD26-Jurkat T cells. Rv-ADA-CD3-IL2sp transduced Jurkat T cells or CD26-Jurkat T cells were cultured for 24 hours and stained with PE-conjugated mAb against ADA and subjected to flow analysis of ADA expression. As shown in FIG. 8C, ADA level on Rv-ADA-CD3-transduced Jurkat T cells was higher than that of Jurkat T cells, indicating CD3-scFv anchored ADA-CD3 on the surface of Jurkat T cells. The ADA level on Rv-ADA-CD3-transduced CD26-Jurkat T cells was further increased compared that on Rv-ADA-CD3-transduced Jurkat T cells, indicating the engagement of ADA and CD26 further enhanced the binding of ADA-CD3 with CD26-Jurkat T cells. Thus, these data suggested that our designed ADA-CD3 can be secreted by and engaged with T cells.

Next, the secreted ADA-CD3's enzyme activity was investigated. Since ADA-CD3 has high binding affinity with T cells and will engage on the surface of the T cells, the T cells were directly used to measure the ADA-CD3's enzyme activity. First, HEK 293 T cells were transduced with Rv-ADA-CD3 and seeded in 96 well plate at different density for overnight. After 24 hours, the cells were counted, and cell density was shown in FIG. 9A. Then same amount of Jurkat T cells was added to the cell culture for 6 hours. The cell culture medium was removed, and the cells were directly subjected to ADA activity assay. As shown in FIG. 9A, Rv-ADA-CD3 resulted in higher enzyme activity of converting ADO to INO at cell culture density of 10e5/well or 5×10e5/well compared to control group, indicating the ADA secretion is stress condition mediated (left: original reads; right: non-transduced HEK 293 T cell background reads were subtracted).

Next, we investigated the binding efficacy of ADA-CD3 on T cells. Jurkat-Dual (NF-KB &IRF/IFN), Jurkat-Dual transduced with Rv-CD26 (Jurkat-Dual-CD26), Jurkat-NFAT or Jurkat-NFAT transduced with Rv-CD26 (Jurkat-NFAT-CD26) reporter cell lines were co-cultured with Rv-ADA or Rv-ADA-CD3 (scFv) transduced 293 T cells for 24 hours. The cell culture medium was then used to measure ADA by ADA activity assay. ADA membrane binding efficiency was calculated using Rv-ADA-CD3-293T/CD26-Jurkat T as maximum and 293T/JurkatT W/O CD26 as background. The results showed that the secreted ADA-CD3 engaged with both Jurkat-Dual and CD26-Jurkat-Dual (FIG. 9B) or Jurkat-NFAT and CD26-Jurkat-NFAT (FIG. 9C) completely, while secreted ADA (without CD3-scFv) only engaged with CD26-Jurkat-Dual (FIG. 9B) or CD26-Jurkat-NFAT (FIG. 9C) at 40% efficacy of secreted ADA-CD3. Thus, these results demonstrated that ADA-CD3 improved engagement of ADA

Next, we investigated if the secreted ADA-CD3 (scFv) can activate human T cells since either CD3-scFv or ADA can provide stimulatory or co-stimulatory signal to human T cells. As shown in FIGS. 10A and 10B, Rv-ADA (ADA1) or Rv-ADA-CD3 (ADA1-CD3) was transduced into 293 T cells. 293 T cell culture medium (supernatant) or 293 T cells were added to the culture of NFAT-luciferase Jurkat reporter cell line. The cells were cultured for 6 hours (FIG. 10A) or 24 hours (FIG. 10B), and then cell culture medium were collected and subjected to luciferase assay. Only Rv-ADA-CD3 transduced 293 T cells induced T cell activation at 24 hours, indicating the secretion of ADA-CD3 is mediated by stress condition (cell culture at high cell density for 24 hours). We also compared Rv-ADA-CD3 and Rv-ADA-CD3-IL2sp. Jurkat NFAT reporter T cells were added with the supernatant of or the Rv-ADA-CD3 or Rv-ADA-CD3 (IL2sp) transduced HEK 293 T cell for 6 hours or 24 hours and subjected to luciferase assay. The results showed that Rv-ADA-CD3-IL2sp induced T cell activation more effectively than Rv-ADA-CD3. ADA-CD3 (IL2sp) expression level were calculated as shown in FIG. 10D. Rv-ADA-CD3 or Rv-ADA-CD3-IL2sp can induce ADA-CD3 expression at 0.3 ug/ml or >0.5 ug/ml at 24 hours individually.

### LDH Cytotoxicity Assay of MR-CAR T Cells

### LDH Assay Protocol

Day 1: In 96 well U-bottom cell culture plate, tumor cells (1×10e4) were co-cultured with effector cells CAR-T and NT-T (non-transduced T cells as negative control) in 200 ul DMEM medium at a gradient E:T ratio as below for 4 h. Maximum load (positive control) well will be added with 20 ul of lysis buffer and culture for 30 minutes.

| E:T ratio | CAR-T (Effector) | Tumor cell (Target) |
|---|---|---|
| 1:1 | 1 × 10e4 | 1 × 10e4 |
| 5:1 | 5 × 10e4 | 1 × 10e4 |
| 10:1 | 1 × 10e5 | 1 × 10e4 |
| 20:1 | 2 × 10e5 | 1 × 10e4 |
| Maximum load | 20 ul of lysis buffer | 1 × 10e4 |
| (positive control) | | |

After 4 hours: Detection of the lactate dehydrogenase (LDH) was conducted as specified by the manufacturer using Enzo LDH cytotoxicity EST assay (Cat #ENZ-KIT157)
   1. The plate will be centrifuge at 250×g for 2 minutes to precipitate the cells.
   2. Transfer 100 ul of cell supernatant to each well of a new flat-bottom optically clear 96-well plate.
   3. Add 100 ul of the working solution to each well.
   4. Protect the plate from light and incubate it at room temperature for 30 minutes.
   5. Add 50 ul of the stop solution to each well.
   6. Measure the absorbance at 490 nm by a microplate reader

The specific lysis was calculated using the following formula:

(experimental-spontaneous release)/(maximum load-spontaneous release)×100%

As shown in FIG. 11, the CAR T cells were co-cultured with HepG2 or Huh7 liver cancer cells and subjected to LDH assay as above. FIG. 11B and FIG. 11C showed that, at 4 hours of co-culture, MR-CAR (GPC3-ADA1-CD3-CD26) T cells displayed enhanced cytotoxicity against HepG2 (FIG. 11B) or Huh7 (FIG. 11C) at E: T ratio of 20:1 compared to that of GPC3-CAR T cells, while no significant difference were observed at E: T ration of 1:1. This is likely due to that the expression and secretion of ADA-CD3 (scFv) needs time. As a result, at the E: T ratio of 1:1, MR-CAR displayed a significantly enhanced cytotoxicity against HepG2 (FIG. 11D) or Huh7 (FIG. 11E) at either 18 hours or 24 hours of co-culture of MR-CAR T cells and the tumor cells. These results indicated that MR-CAR T cells displayed enhanced cytotoxicity against specific tumor cells in vitro.

### In Vivo Mouse Studies of MR-CAR T Cell Therapy

6 weeks old NSG mice were purchased from Jax laboratory, and each group has 5 mice. To investigate the effects of MR-CAR T cell therapy in liver cancer, groups of NSG mice were first subcutaneously inoculated with 2×10e6 Huh7 HCC tumor cells in 200 ul medium on the right flank at day 0. At day 7, the Huh7 tumor bearing mice were treated through tail vein with 200 ul PBS, 2×10e6 GPC3-CAR T cells in 200 ul medium, or 2×10e6 GPC3-ADA1-CD3-CD26-CAR T cells in 200 ul medium (designated as GPC3-MR-CAR). The mice were monitored every 2 or 3 days. Tumor size were measure with caliper and the tumor size was calculated as: tumor size=L×W×W/2 (L: length of the tumor, W: width of the tumor). The mice body weight was also measured to monitor the toxicity of MR-CAR T cell therapy.

To investigate the effects of MR-CAR T cell therapy in lung cancer, groups of NSG mice were first subcutaneously inoculated with 2×10e6 A549 NSCLC tumor cells in 200 ul medium on the right flank at day 0. At day 7, the A549 tumor bearing mice were treated through tail vein with 2×10e6 HER2-CAR T cells, or 2×10e6 HER2-ADA1-CD3-CD26-CAR T cells in 200 ul medium (designated as HER2-MR-CAR) or no treatment as control (NT). The tumor size and body weight were measured as described above.

In both models, MR-CAR T cells showed enhanced anti-tumor activity. MR-CAR T cells significantly inhibit tumor growth in both Huh7 HCC or A549 NSCLC mouse models while original CAR T cells only moderately inhibit Huh7 or A549 tumor growth. In addition, no significant difference in body weight was observed in both Huh7 HCC and A549 NSCLC mouse models, indicating MR-CAR T cells didn't induce toxicity in these mouse models.

The term “substantially” is defined as largely but not necessarily wholly what is specified, as understood by a person of ordinary skill in the art. In any disclosed embodiment, the terms “substantially”, “approximately”, “generally”, and “about” may be substituted with “within [a percentage] of” what is specified, where the percentage includes 0.1, 1, 5, and 10 percent.

The foregoing outlines features of several embodiments so that those skilled in the art may better understand the aspects of the disclosure. Those skilled in the art should appreciate that they may readily use the disclosure as a basis for designing or modifying other processes and structures for carrying out the same purposes and/or achieving the same advantages of the embodiments introduced herein. Those skilled in the art should also realize that such equivalent constructions do not depart from the spirit and scope of the disclosure, and that they may make various changes, substitutions, and alterations herein without departing from the spirit and scope of the disclosure. The scope of the invention should be determined only by the language of the claims that follow. The term “comprising” within the claims is intended to mean “including at least” such that the recited listing of elements in a claim are an open group. The terms “a”, “an”, and other singular terms are intended to include the plural forms thereof unless specifically excluded.

1-92. (canceled)

93. An engineered cell that expresses a tumor-specific chimeric antigen receptor (CAR) and an Adenosine deaminase (ADA).

94. The cell of claim 93, wherein the ADA is ADA 1 or ADA2.

95. The cell of claim 93, wherein the ADA is a secreted protein or a membrane-bound protein.

96. The cell of claim 93, wherein the ADA is operably linked to one or more of a linker sequence, a human CD3 specific ligand, antibody, scFv, or its derivates.

97. The cell of claim 96, further comprising IL2 signal peptide that is operably linked to the ADA.

98. The cell of claim 93 further comprising human CD26 or its derivates.

99. A pharmaceutical composition comprising the engineered cell of claim 93 and a pharmaceutically acceptable carrier.

100. A method of treatment, wherein the method comprises administering the engineered cells of claim 93 to a patient in an amount effective for treatment of a disease.

101. The method of claim 100, wherein the treatment involves immunomodulation of T-cells.

102. The method of claim 100, wherein the disease is cancer.

103. A recombinant protein comprising an amino acid sequence encoding an amino deaminase (ADA) that is operably linked to one or more of a linker sequence, a human CD3 specific ligand, antibody, scFv, or its derivates.

104. The recombinant protein of claim 103, wherein the ADA is ADA1 or ADA2.

105. A pharmaceutical composition comprising the recombinant protein of claim 104 and a pharmaceutically acceptable carrier.

106. A method of administering the recombinant protein of claim 104 to a patient in an amount effective for treatment of a disease.

107. The method of claim 106, wherein the treatment involves immunomodulation of T-cells.

108. The method of claim 106, wherein the disease is cancer.


SEQUENCE LISTING (32 sequences)

SEQ ID NO: 1 - protein, 1837 aa, synthetic construct
MDWIWRILFLVGAATGAHSEVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIKALDLEPKSCDKTHTCPPCPDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMRRMQLLLLIALSLALVTNSMAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGSGATNFSLLKQAGDVEENPGPMKTPWKVLLGLLGAAALVTIITVPVVLLNKGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 2 - protein, 1965 aa, synthetic construct
MDWIWRILFLVGAATGAHSEVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIKALDLEPKSCDKTHTCPPCPDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRARLLPVYELSGEHHDEEWSVKTYQEVAQKFVETHPEFIGIKIIYSDHRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGSGATNFSLLKQAGDVEENPGPMKTPWKVLLGLLGAAALVTIITVPVVLLNKGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 3 - protein, 1049 aa, synthetic construct
MDWIWRILFLVGAATGAHSEVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIKALDLEPKSCDKTHTCPPCPDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMRRMQLLLLIALSLALVTNSMAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 4 - protein, 1177 aa, synthetic construct
MDWIWRILFLVGAATGAHSEVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIKALDLEPKSCDKTHTCPPCPDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRARLLPVYELSGEHHDEEWSVKTYQEVAQKFVETHPEFIGIKIIYSDHRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATK

SEQ ID NO: 5 - protein, 1170 aa, synthetic construct
MRRMQLLLLIALSLALVTNSMAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLGSGEGRGSLLTCGDVEENPGPMKTPWKVLLGLLGAAALVTIITVPVVLLNKGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 6 - protein, 1298 aa, synthetic construct
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRARLLPVYELSGEHHDEEWSVKTYQEVAQKFVETHPEFIGIKIIYSDHRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKGSGEGRGSLLTCGDVEENPGPMKTPWKVLLGLLGAAALVTIITVPVVLLNKGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 7 - protein, 383 aa, synthetic construct
MRRMQLLLLIALSLALVTNSMAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 8 - protein, 511 aa, synthetic construct
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRARLLPVYELSGEHHDEEWSVKTYQEVAQKFVETHPEFIGIKIIYSDHRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATK

SEQ ID NO: 9 - protein, 766 aa, synthetic construct
MKTPWKVLLGLLGAAALVTIITVPVVLLNKGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 10 - protein, 1012 aa, synthetic construct
MDWIWRILFLVGAATGAHSEVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIKALDLEPKSCDKTHTCPPCPDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSSMAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLSSDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 1160 aa, synthetic construct
MDWIWRILFLVGAATGAHSEVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIKALDLEPKSCDKTHTCPPCPDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSSMLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRARLLPVYELSGEHHDEEWSVKTYQEVAQKFVETHPEFIGIKIIYSDHRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKSSDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 2022 aa, synthetic construct
MDWIWRILFLVGAATGAHSEVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIKALDLEPKSCDKTHTCPPCPDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRRMQLLLLIALSLALVTNSMAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLSSDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMKTPWKVLLGLLGAAALVTIITVPVVLLNKGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 13 - protein, 2022 aa, synthetic construct; identical to SEQ ID NO: 12

SEQ ID NO: 14 - protein, 1355 aa, synthetic construct
MRRMQLLLLIALSLALVTNSMAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLSSDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMKTPWKVLLGLLGAAALVTIITVPVVLLNKGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 15 - protein, 1483 aa, synthetic construct
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRARLLPVYELSGEHHDEEWSVKTYQEVAQKFVETHPEFIGIKIIYSDHRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKSSDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMKTPWKVLLGLLGAAALVTIITVPVVLLNKGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 16 - protein, 568 aa, synthetic construct
MRRMQLLLLIALSLALVTNSMAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLSSDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 696 aa, synthetic construct
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRARLLPVYELSGEHHDEEWSVKTYQEVAQKFVETHPEFIGIKIIYSDHRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKSSDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 2085 aa, synthetic construct
MDWIWRILFLVGAATGAHSEVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIKALDLEPKSCDKTHTCPPCPDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMRRMQLLLLIALSLALVTNSMAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKSGSGEGRGSLLTCGDVEENPGPMKTPWKVLLGLLGAAALVTIITVPVVLLNKGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 19 - protein, 2233 aa, synthetic construct
MDWIWRILFLVGAATGAHSEVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIKALDLEPKSCDKTHTCPPCPDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPGSGEGRGSLLTCGDVEENPGPMLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRARLLPVYELSGEHHDEEWSVKTYQEVAQKFVETHPEFIGIKIIYSDHRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKSGSGEGRGSLLTCGDVEENPGPMKTPWKVLLGLLGAAALVTIITVPVVLLNKGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 20 - protein, 1297 aa, synthetic construct
MDWIWRILFLVGAATGAHSEVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIKALDLEPKSCDKTHTCPPCPDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMRRMQLLLLIALSLALVTNSMAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKS

SEQ ID NO: 21 - protein, 1424 aa, synthetic construct
MDWIWRILFLVGAATGAHSEVQLQQSGPELKKPGETVKISCKASGYPFTNYGMNWVKQAPGQGLKWMGWINTSTGESTFADDFKGRFDFSLETSANTAYLQINNLKSEDMATYFCARWEVYHGYVPYWGQGTTVTVSSGGGGSGGGGSGGGGSDIQLTQSHKFLSTSVGDRVSITCKASQDVYNAVAWYQQKPGQSPKLLIYSASSRYTGVPSRFTGSGSGPDFTFTISSVQAEDLAVYFCQQHFRTPFTFGSGTKLEIKALDLEPKSCDKTHTCPPCPDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRARLLPVYELSGEHHDEEWSVKTYQEVAQKFVETHPEFIGIKIIYSDHRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKRWDKFIADVATKSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKS

SEQ ID NO: 22 - protein, 1418 aa, synthetic construct
MRRMQLLLLIALSLALVTNSMAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKSGSGEGRGSLLTCGDVEENPGPMKTPWKVLLGLLGAAALVTIITVPVVLLNKGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 23 - protein, 1545 aa, synthetic construct
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRARLLPVYELSGEHHDEEWSVKTYQEVAQKFVETHPEFIGIKIIYSDHRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEADAATYYCQQWSSNPLTFGAGTKLELKSGSGEGRGSLLTCGDVEENPGPMKTPWKVLLGLLGAAALVTIITVPVVLLNKGTDDATADSRKTYTLTDYLKNTYRLKLYSLRWISDHEYLYKQENNILVFNAEYGNSSVFLENSTFDEFGHSINDYSISPDGQFILLEYNYVKQWRHSYTASYDIYDLNKRQLITEERIPNNTQWVTWSPVGHKLAYVWNNDIYVKIEPNLPSYRITWTGKEDIIYNGITDWVYEEEVFSAYSALWWSPNGTFLAYAQFNDTEVPLIEYSFYSDESLQYPKTVRVPYPKAGAVNPTVKFFVVNTDSLSSVTNATSIQITAPASMLIGDHYLCDVTWATQERISLQWLRRIQNYSVMDICDYDESSGRWNCLVARQHIEMSTTGWVGRFRPSEPHFTLDGNSFYKIISNEEGYRHICYFQIDKKDCTFITKGTWEVIGIEALTSDYLYYISNEYKGMPGGRNLYKIQLSDYTKVTCLSCELNPERCQYYSVSFSKEAKYYQLRCSGPGLPLYTLHSSVNDKGLRVLEDNSALDKMLQNVQMPSKKLDFIILNETKFWYQMILPPHFDKSKKYPLLLDVYAGPCSQKADTVFRLNWATYLASTENIIVASFDGRGSGYQGDKIMHAINRRLGTFEVEDQIEAARQFSKMGFVDNKRIAIWGWSYGGYVTSMVLGSGSGVFKCGIAVAPVSRWEYYDSVYTERYMGLPTPEDNLDHYRNSTVMSRAENFKQVEYLLIHGTADDNVHFQQSAQISKALVDVGVDFQAMWYTDEDHGIASSTAHQHIYTHMSHFIKQCFSLP

SEQ ID NO: 24 - protein, 631 aa, synthetic construct
MRRMQLLLLIALSLALVTNSMAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNLSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKS

SEQ ID NO: 25 - protein, 759 aa, synthetic construct
MLVDGPSERPALCFLLLAVAMSFFGSALSIDETRAHLLLKEKMMRLGGRLVLNTKEELANERLMTLKIAEMKEAMRTLIFPPSMHFFQAKHLIERSQVFNILRMMPKGAALHLHDIGIVTMDWLVRNVTYRPHCHICFTPRGIMQFRFAHPTPRPSEKCSKWILLEDYRKRVQNVTEFDDSLLRNFTLVTQHPEVIYTNQNVVWSKFETIFFTISGLIHYAPVFRDYVFRSMQEFYEDNVLYMEIRARLLPVYELSGEHHDEEWSVKTYQEVAQKFVETHPEFIGIKIIYSDHRSKDVAVIAESIRMAMGLRIKFPTVVAGFDLVGHEDTGHSLHDYKEALMIPAKDGVKLPYFFHAGETDWQGTSIDRNILDALMLNTTRIGHGFALSKHPAVRTYSWKKDIPIEVCPISNQVLKLVSDLRNHPVATLMATGHPMVISSDDPAMFGAKGLSYDFYEVFMGIGGMKADLRTLKQLAMNSIKYSTLLESEKNTFMEIWKKRWDKFIADVATKSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKS

SEQ ID NO: 26 - protein, 71 aa, synthetic construct
DPKFWVLVVVGGVLACYSLLVTVAFIIFWVRSKRSRLLHSDYMNMTPRRPGPTRKHYQPYAPPRDFAAYRS

SEQ ID NO: 27 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 15 aa, synthetic construct
GGGGSGGGGSGGGGS

SEQ ID NO: 29 - protein, 241 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKS

SEQ ID NO: 30 - protein, 21 aa, synthetic construct
GSGEGRGSLLTCGDVEENPGP

SEQ ID NO: 31 - protein, 22 aa, synthetic construct
GSGATNFSLLKQAGDVEENPGP

SEQ ID NO: 32 - protein, 20 aa, synthetic construct
MRRMQLLLLIALSLALVTNS